(19) United States
(12) Patent Application Publication
(10) Pub. No.: US 2022/0387543 A1
(43) Pub. Date: Dec. 8, 2022

(54) SORE THROAT REMEDY

(71) Applicant: Shaun Morgan Welgemoed, Woodland Hills, CA (US)

(72) Inventor: Shaun Morgan Welgemoed, Woodland Hills, CA (US)

(21) Appl. No.: 17/825,831

(22) Filed: May 26, 2022

Related U.S. Application Data
(60) Provisional application No. 63/193,369, filed on May 26, 2021.

(51) Int. Cl. A61K 36/906; A61P 11/04; A61K 9/00; A61K 47/42; A61K 35/644; A61K 36/752

(52) U.S. Cl. CPC A61K 36/906; A61P 11/04; A61K 9/0095; A61K 47/42; A61K 35/644; A61K 36/752

(57) ABSTRACT

A method of making a sore throat remedy. The method includes providing beef gelatin powder, providing honey powder, providing lemon powder, providing ginger powder, and combining the beef gelatin powder, honey powder, lemon powder, and ginger powder with water for consumption.

### CROSS-REFERENCE TO RELATED APPLICATIONS

This application claims priority to U.S. Provisional Application No. 63/193,369, filed May 26, 2021, the entirety of which is incorporated by reference herein.

### BACKGROUND

The background description provided herein is for the purpose of generally presenting the context of the disclosure. The work of the presently named inventors, to the extent it is described in this background section, as well as aspects of the description that may not otherwise qualify as prior art at the time of filing, are neither expressly nor impliedly admitted as prior art against the present disclosure.

Those whose professions or other activities may rely on frequent and/or strenuous use of their voice may often suffer from a sore or irritated throat that could hinder or prevent ongoing work. In some scenarios, such as with some singers, voice actors, public speakers, professors, teachers, etc., sore throat sufferers may not have the luxury of resting their voice due to scheduling or other requirements. Existing sore throat remedies suffer from several shortcomings, including unhealthy ingredients, inadequate sore throat relief, undesirable flavor or texture, etc. A sore throat remedy is needed that cures one or all of these shortcomings.

### SUMMARY

The following presents a simplified summary of the present disclosure in order to provide a basic understanding of some aspects of the disclosure. This summary is not an extensive overview of the disclosure. It is not intended to identify key or critical elements of the disclosure or to delineate the scope of the disclosure. The following summary merely presents some concepts of the disclosure in a simplified form as a prelude to the more detailed description provided below.

In one embodiment, the disclosure describes a powdered composition for mixing with water to provide a sore throat remedy. The composition may include between about 30 percent and about 35 percent beef gelatin powder by volume, between about 40 percent and about 45 percent honey powder by volume, between about 15 percent and about 20 percent lemon powder by volume, and between about 5 percent and about 10 percent ginger powder by volume.

In another embodiment, the disclosure describes a liquid sore throat remedy. The liquid sore throat remedy may include between about 4 percent and about 6 percent beef gelatin concentration by volume, between about 6 percent and about 8 percent honey concentration by volume, between about 2 percent and about 4 percent lemon concentration by volume, between about 0.5 percent and about 2.5 percent ginger concentration by volume, and between about 80 percent and about 85 percent water by volume.

In another embodiment, the disclosure describes a method of making a sore throat remedy. The method may include providing beef gelatin powder, providing honey powder, providing lemon powder, providing ginger powder, and combining the beef gelatin powder, honey powder, lemon powder, and ginger powder with water.

These and other features, aspects and advantages of the present invention will become better understood with reference to the following description and claims.

### DETAILED DESCRIPTION

The present disclosure now will be described more fully hereinafter with reference to the accompanying drawings, which form a part hereof, and which show, by way of illustration, specific exemplary embodiments by which the disclosure may be practiced. This disclosure may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as methods or devices. Accordingly, the present invention may take the form of an entirely hardware embodiment, an entirely software embodiment or an embodiment combining software and hardware aspects. The following detailed description is, therefore, not to be taken in a limiting sense.

Throughout the specification and claims, the following terms take the meanings explicitly associated herein, unless the context clearly dictates otherwise. The phrase “in one embodiment” as used herein does not necessarily refer to the same embodiment, although it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and includes plural references. The meaning of “in” includes “in” and “on.”

In some embodiments, the composition described in the present disclosure may be used as a remedy and/or prevent sore, strained, fatigued, or otherwise irritated throats. In some embodiments, the composition may be an all-natural remedy that may include only natural ingredients or ingredients derived directly from natural ingredients. The composition may be particularly useful in treating sore throats suffered by singers, orators, public speakers, stage actors, teachers, voice actors, or other people whose occupation, hobby, or other activities that rely significantly on their voice and voice usage. In some embodiments, the disclosed composition may be used prior to voice use in order to prevent damage or further damage to the throat, larynx, vocal cords, etc. In some embodiments, the disclosed composition may be used to sooth the throat, larynx, vocal cords, etc., after they may have already been damaged from use or overuse. For example, singers who may be recording music or be giving frequent live performances may experience a sore throat and/or vocal cords but may not be able to rest their voice due to studio time schedules, touring, or other live performance schedules, etc. In such scenarios, the disclosed composition consumed in its liquid or other forms may provide for sore throat relief and may allow the singer or other user to continue recording and/or performing without discomfort or with reduced discomfort.

The disclosed sore throat remedy may be provided in various liquid or solid forms. In some embodiments, the sore throat remedy may be in a powdered form that may be mixed or dissolved in water or another liquid to be swallowed, swished or gargled in the mouth, or otherwise ingested by a user. In some embodiments, the powdered form of the disclosed composition may be packaged in individual dose blister packs, in bulk containers for multiple doses, individual or multi-dose pouches or packets, or in any other suitable packaging. In some embodiments, the sore throat remedy may be supplied in solid form, such as a tablet that may be dissolved in liquid prior to consumption, in a lozenge or other hard candy that may be dissolved in a user's mouth, in an edible gummy, etc. Such solid forms may be packaged in any of a number of ways, such as individually, in bulk packaging, etc. In some embodiments, the sore throat remedy may be provided in a liquid form, such as in bottles, cans, jars, or other suitable packaging, and may be consumed directly, or provided in a liquid form to be used as a spray or other atomized form. In some embodiments, the composition may be provided in a pre-measured dosage amount in a container that may be introduced into a drink machine, such as a single- or multiple-serving coffee or tea brewing machine. In such embodiments, the container may be configured to be pierced so that water or another mixing liquid may flow through the packaging and into a serving container for consumption.

In some embodiments, the sore throat remedy may comprise four primary components: (1) beef gelatin powder, (2) honey powder, (3) lemon powder, and (4) ginger powder. In some embodiments, each component may be made from organic ingredients and may include substantially no or limited extraneous and/or unnecessary ingredients such as preservatives, unwanted chemicals, etc. Those skilled in the art will understand that certain additional components may also be included in the sore throat remedy without straying from the scope of the disclosure, and that some embodiments may include fewer than all of the ingredients listed. In some embodiments, a combination of these and potentially other dry ingredients may be blended together or otherwise packaged together in single or multiple dose measurements in the ratios described in more detail herein. In the powdered form, a liquid such as water may be mixed with the powdered composition and the powder may substantially (or completely) dissolve in the liquid. In some embodiments, the ingredients described herein may be formed into a dissolvable tablet or other solid form, that may be mixed with liquid or ingested directly. In some embodiments, a the ingredients may be formed into a concentrated liquid form that may be combined with another liquid prior to consumption. It is contemplated that binders or other ingredients may be included in order to prepare the composition into one of multiple consumable forms.

Beef gelatin may be a protein supplement derived from bovine collagen and may be provided in powdered or granular form. In some embodiments, beef gelatin may be a mixture of peptides and proteins produced by partial hydrolysis of collagen extracted from animal tissue. Those skilled in the art may recognize that beef gelatin may also be referred to as hydrolyzed collagen, collagen hydrolysate, gelatine hydrolysate, or hydrolyzed gelatin. Beef gelatin may contain absorbable protein, amino acids, may be low in calories, sugar, fat, and carbohydrates. In some embodiments, beef gelatin may be nearly tasteless and odorless with a substantially colorless appearance. When dry, beef gelatin powder may be brittle and may allow for dry mixing and storage with other dried and/or powdered ingredients. When moist, beef gelatin may take on a gummy or viscous texture that, for example, may be beneficial for coating a user's throat, larynx, vocal cords, etc. that may assist in providing pain relief and/or tissue protection. Beef gelatin may be dissolvable in certain liquids, such as water, particularly warm or hot water. In some embodiments, beef gelatin powder may have a density of between about 0.2 g/mL and about 0.3 g/mL, or about 0.211 g/mL in other embodiments. Due to the protein content from the beef gelatin powder, in some embodiments, the disclosed composition may be used as a protein supplement on its own, mixed with water, or mixed with other foods such as shakes or smoothies.

In some embodiments, honey powder may be made from dehydrating honey until substantially all the honey's moisture has evaporated. The resulting solid may be ground into a fine powder. In some embodiments, the pure honey powder may be mixed with one or more stabilizers, such as starch or sugar, in order to maintain a powder or granular form. Stabilizers may help prevent the pure honey powder from absorbing humidity in the air, which may make the honey powder sticky. In some embodiments, the honey powder may be made from organic honey. In some embodiments, the density of honey powder may be between about 0.4 g/mL and about 0.7 g/mL, or between about 0.48 g/mL and about 0.71 g/mL in other embodiments.

In some embodiments, lemon powder may be made from dried or dehydrated pulp of lemons. After the drying process, the dried pulp may be finely ground into granules or powder. In some embodiments, lemon powder may be made from lemon peals that have been dried or dehydrated and subsequently crushed, ground, and/or churned into granules or powder. In some embodiments, lemon powder may be made from a combination of dried pulp granules and crushed lemon peals. In some embodiments, the lemon powder may be made from organic lemons. In some embodiments, the density of lemon powder may be between about 0.7 g/mL and about 0.8 g/mL, or about 0.77 g/mL in other embodiments.

In some embodiments, ginger powder may also be referred to as ground ginger. The ginger powder may be made by drying out or dehydrating ginger root and grinding the dried ginger root into granules or a fine powder. In some embodiments, the ginger root may be peeled and sliced before drying or dehydrating. In some embodiments, the ginger powder may be made from ginger pulp that may be dried or dehydrated and subsequently crushed or ground into granules or powder. In some embodiments, the ginger powder may be made from organic ginger root. In some embodiments, the density of ginger powder may be between about 0.3 g/mL and about 0.4 g/mL, and about 0.35 g/mL in other embodiments.

The disclosed sore throat remedy may include the ingredients described above in particular ratios to one another. In some embodiments, the sore throat remedy may be a solid and/or powdered composition including beef gelatin powder at about 32.4 percent by volume, honey powder at about 43.2 percent by volume, lemon powder at about 16.2 percent by volume, and ginger powder at about 8.1 percent by volume. The powdered composition may include beef gelatin powder between about 13 percent by weight and about 16 percent by weight, honey powder between about 48 percent by weight and 58 percent by weight, lemon powder between about 24 percent by weight and 29 percent by weight, and ginger powder between about 5 percent by weight and about 7 percent by weight. The disclosed powdered form of the sore throat remedy may be combined with and/or dissolved in water prior to consumption. Table 1 includes one range of approximate percent composition by volume and composition by weight of some embodiments of a powdered form of the sore throat remedy:

TABLE 1

| Ingredient | % by volume | % by weight |
|---|---|---|
| Beef Gelatin Powder | 30-35% | 12-17% |
| Honey Powder | 40-45% | 45-60% |
| Lemon Powder | 15-20% | 20-30% |
| Ginger Powder | 5-10% | 5-10% |

In some embodiments, the powdered composition may include beef gelatin powder between about 25 percent by weight and about 35 percent by weight, between about 30 percent by weight and about 33 percent by weight, or about 32.5 percent by weight. The composition may include honey powder between about 40 percent by weight and about 50 percent by weight, between about 42 percent by weight and about 45 percent by weight, or about 43.2 percent by weight. The composition may include lemon powder between about 10 percent by weight and 20 percent by weight, between about 15 percent by weight and about 18 percent by weight, or about 16.2 percent by weight. The composition may include ginger powder between about 5 percent by weight and about 10 percent by weight, between about 7 percent by weight and about 9 percent by weight, or about 8.1 percent by weight. The disclosed powdered form of the sore throat remedy may be combined with and/or dissolved in water prior to consumption. Table 2 includes one range of approximate percent composition by volume and composition by weight of some embodiments of a powdered form of the sore throat remedy:

TABLE 2

| Ingredient | % by weight |
|---|---|
| Beef Gelatin Powder | 25-35% |
| Honey Powder | 40-50% |
| Lemon Powder | 10-20% |
| Ginger Powder | 5-10% |

In some embodiments, the sore throat remedy may be provided as a liquid in which the ingredients above may be dissolved in water. In such embodiments, the liquid composition may include beef gelatin powder present in a concentration of between about 4-6 percent by volume or about 5.2 percent by volume, honey powder present in a concentration of about 6-8 percent by volume or about 7 percent by volume, lemon powder present in a concentration of about 1.5-3.5 percent by volume or about 2.6 percent by volume, ginger powder present in a concentration of about 0.5 to about 2.5 percent by volume or about 1.3 percent by volume, and water present in a concentration of about 80-85 percent by volume or about 83.8 percent by volume. The liquid composition may include beef gelatin at about 1-2 percent by weight or about 1.2 percent by weight, honey powder between about 3.7 percent by weight and about 5.4 percent by weight, lemon powder between about 1.5-2.5 percent by weight or at about 2.2 percent by weight, ginger powder between about 0.2-1.5 percent by weight or at about 0.5 percent by weight, and water at or between about 90.8 percent by weight and about 92.4 percent by weight. In some embodiments, the water may be at a temperature suitable for one or more of the powdered ingredients to readily dissolve, such as at or between about 90 degrees Fahrenheit and about 110 degrees Fahrenheit, or at least about 100 degrees Fahrenheit, either immediately or upon stirring or other agitation. Table 3 includes one range of approximate percent concentration by volume and concentration by weight of some embodiments of a liquid form of the sore throat remedy:

TABLE 3

| Ingredient | % by volume | % by weight |
|---|---|---|
| Beef Gelatin Powder | 4-6% | 0.5-2.5% |
| Honey Powder | 6-8% | 3-6% |
| Lemon Powder | 2-4% | 1-3% |
| Ginger Powder | 0.5-2.5% | 0.2-0.8% |
| Water | 80-85% | 88-93% |

In some embodiments, the disclosure describes methods for preparing the sore throat remedy described herein. In some embodiments, the method of making a sore throat remedy may include providing beef gelatin powder, providing honey powder, providing lemon powder, providing ginger powder, and combining the beef gelatin powder, the honey powder, the lemon powder, and the ginger powder with water. In some embodiments, the method may include combining the beef gelatin powder, the honey powder, the lemon powder, and the ginger powder with water until the powdered ingredients are substantially dissolved. In some embodiments, the method may include heating the water to at least about 90 degrees Fahrenheit, or about 100 degrees Fahrenheit (37.8 degrees Celsius). In some embodiments, the method may include using about beef gelatin powder that may have a volume of about 7.4 mL, honey powder that may have a volume of about 9.9 mL, lemon powder that may have a volume of about 3.7 mL, and ginger powder that may have a volume of about 1.85 mL. In some embodiments, the water may have a volume of about 118.3 mL. In some embodiments, the method may include using beef gelatin powder that may have a weight of about 7 grams, honey powder that may have a weight of about 9.5 grams, lemon powder that may have a weight of about 3.5 grams, and ginger powder that may have a weight of about 1.75 grams. In some embodiments, the method may include using organic beef gelatin powder, organic honey powder, organic lemon powder, and organic ginger powder.

In some embodiments, the method may include preparing the combined powdered ingredients into one or more deliverable forms. For example, the beef gelatin powder, the honey powder, the lemon powder, and the ginger powder may be combined to form a powder composition. The powder composition may be packaged into blister packs or other bulk and/or individual packaging, or may be formed into a dissolvable tablet, a lozenge, an edible gummy, etc. In some embodiments, the powder composition may be formed into a dissolvable tablet by compressing the powder composition such as in a mold. Forming the dissolvable tablet may include adding trace amounts of liquid such as water or other binding aids. Forming the dissolvable tablet may also include applying heat to the powder composition. In some embodiments, a lozenge or edible gummy may be formed from the powder composition such as by using methods known in the art.

### EXAMPLES

### Example A

In Example A, a single dose of powdered sore throat remedy was prepared using 0.5 tablespoons (1.5 teaspoons or 7.4 mL) of beef gelatin powder, 2 teaspoons (9.9 mL) of honey powder, 0.75 teaspoons (3.7 mL) of lemon powder, and 0.375 teaspoons (1.85 mL) of ginger powder. The concentration of beef gelatin powder by volume in Example A is about 32.4 percent by volume, the concentration of honey powder is about 43.2 percent by volume, the concentration of lemon powder is about 16.2 percent by volume, and the concentration of ginger powder is about 8.1 percent by volume. In Example A, the powdered composition was combined with about 4 fluid ounces of water at least about 100 degrees Fahrenheit (37.8 degrees Celsius). The powdered composition combined with water was reported to have a pleasant taste and high effectiveness for soothing and/or protecting the user's throat, larynx, and vocal cords.

### Example B

In Example B, a single dose of liquid sore throat remedy was prepared using 0.5 tablespoons (1.5 teaspoons or 7.4 mL) of beef gelatin powder, 2 teaspoons (9.9 mL) of honey powder, 0.75 teaspoons (3.7 mL) of lemon powder, 0.375 teaspoons (1.85 mL) of ginger powder, and about 4 fluid ounces (118.3 mL) of water. The concentration of beef gelatin by volume in Example B is about 5.2 percent by volume, the concentration of honey powder is about 7 percent by volume, the concentration of lemon powder is about 2.6 percent by volume, the concentration of ginger powder is about 1.3 percent by volume, and the concentration of water is about 84 percent by volume. The powdered composition combined with water was reported to have a pleasant taste and high effectiveness for soothing and/or protecting the user's throat, larynx, and vocal cords.

### Example C

In Example C, a single dose of powdered sore throat remedy was prepared using about 7 grams (or 7.09 grams) of beef gelatin powder, about 9.5 grams (or 9.44 grams) of honey powder, about 3.5 grams (or 3.54 grams) of lemon powder, and about 1.75 grams (or 1.77 grams) of ginger powder. The concentration of beef gelatin powder by weight in Example C is about 32.5 percent by weight, the concentration of honey powder is about 43.2 percent by weight, the concentration of lemon powder is about 16.2 percent by volume, and the concentration of ginger powder is about 8.1 percent by volume. In Example C, the powdered composition was combined with about 4 fluid ounces of water at least about 100 degrees Fahrenheit (37.8 degrees Celsius). The powdered composition combined with water was reported to have a pleasant taste and high effectiveness for soothing and/or protecting the user's throat, larynx, and vocal cords.

In view of the above, it will be seen that the several advantages of the disclosure are achieved and other advantageous results attained. As various changes could be made in the above compositions and methods without departing from the scope of the disclosure, it is intended that all matter contained in the above description and shown in any accompanying drawings shall be interpreted as illustrative and not in a limiting sense.

While various embodiments have been described above, it should be understood that such disclosures have been presented by way of example only, and are not limiting. Thus, the breadth and scope of the subject methods, compositions, and systems should not be limited by any of the above-described exemplary embodiments, but should be defined only in accordance with the following claims and their equivalents.

Having now fully described the subject methods, compositions, and systems, it will be understood by those of ordinary skill in the art that the same can be performed within a wide and equivalent range of conditions, formulations and other parameters without affecting their scope or any embodiment thereof. All cited patents, patent applications, and publications are fully incorporated by reference in their entirety.

1. A powdered composition for mixing with water, the composition comprising:
between about 30 percent and about 35 percent beef gelatin powder by volume;
between about 40 percent and about 45 percent honey powder by volume;
between about 15 percent and about 20 percent lemon powder by volume; and
between about 5 percent and about 10 percent ginger powder by volume.

2. The powdered composition of claim 1, wherein a volume of beef gelatin powder is about 7.4 mL, a volume of honey powder is about 9.9 mL, a volume of lemon powder is about 3.7 mL, and a volume of ginger power is about 1.85 mL.

3. The powdered composition of claim 1, wherein the beef gelatin powder is organic beef gelatin powder, the honey powder is organic honey powder, the lemon powder is organic lemon powder, and the ginger powder is organic ginger powder.

4. The powdered composition of claim 1, wherein the composition comprises:
about 32.4 percent beef gelatin powder by volume;
about 43.2 percent honey powder by volume;
about 16.2 percent lemon powder by volume; and
about 8.4 percent ginger powder by volume.

5. The powdered composition of claim 1, wherein the composition comprises about 7 grams of beef gelatin, about 9.5 grams of honey powder, about 3.5 grams of lemon powder, and about 1.75 grams of ginger powder.

6. A liquid sore throat remedy comprising:
between about 4 percent and about 6 percent beef gelatin concentration by volume;
between about 6 percent and about 8 percent honey concentration by volume;
between about 2 percent and about 4 percent lemon concentration by volume;
between about 0.5 percent and about 2.5 percent ginger concentration by volume; and
between about 80 percent and about 85 percent water by volume.

7. The liquid sore throat remedy of claim 6, wherein a volume of beef gelatin is about 7.4 mL, a volume of honey powder is about 9.9 mL, a volume of lemon powder is about 3.7 mL, a volume of ginger power is about 1.85 mL, and a volume of water is about 118.3 mL.

8. The liquid sore throat remedy of claim 6, wherein the beef gelatin is organic beef gelatin, the honey is organic honey, the lemon is organic lemon, and the ginger is organic ginger.

9. A method of making a sore throat remedy, the method comprising:
providing beef gelatin powder;
providing honey powder;
providing lemon powder;
providing ginger powder; and
combining the beef gelatin powder, the honey powder, the lemon powder, and the ginger powder with water.

10. The method of claim 9 further comprising combining the beef gelatin powder, the honey powder, the lemon powder, and the ginger powder with water until each are substantially dissolved.

11. The method of claim 9 further comprising heating the water to at least about 100 degrees Fahrenheit (37.8 degrees Celsius).

12. The method of claim 9, wherein:
the beef gelatin powder has a volume of about 7.4 mL;
the honey powder has a volume of about 9.9 mL;
the lemon powder has a volume of about 3.7 mL;
the ginger powder has a volume of about 1.85 mL; and
the water has a volume of about 118.3 mL.

13. The method of claim 12 further comprising heating the water to at least about 100 degrees Fahrenheit (37.8 degrees Celsius).

14. The method of claim 9, wherein:
the beef gelatin powder has a weight of about 7 grams;
the honey powder has a weight of about 9.5 grams;
the lemon powder has a weight of about 3.5 grams; and
the ginger powder has a weight of about 1.75 grams.

15. The method of claim 9, wherein the beef gelatin powder is organic beef gelatin powder, the honey powder is organic honey powder, the lemon powder is organic lemon powder, and the ginger powder is organic ginger powder.

16. The method of claim 9 further comprising:
combining the beef gelatin powder, the honey powder, the lemon powder, and the ginger powder together to form a powder composition; and
compressing the powder composition into a dissolvable tablet.

17. The method of claim 16, wherein the powder composition is compressed in a mold.
